(19) United States
(12) Patent Application Publication
(10) Pub. No.: US 2026/0232571 A1
(43) Pub. Date: Aug. 13, 2026

(54) FORMULATIONS FOR THE TREATMENT OF SKIN DISEASES AND CONDITIONS

(71) Applicant: Keylika, Inc., Walnut Creek, CA (US)

(72) Inventor: Buddhadev P. Chaudhuri, Walnut Creek, CA (US)

(73) Assignee: Keylika, Inc., Walnut Creek, CA (US)

(21) Appl. No.: 19/156,511

(22) PCT Filed: Feb. 14, 2024
(86) PCT No.: PCT/US2024/015837

§ 371 Date: Aug. 14, 2025

Related U.S. Application Data
(60) Provisional application No. 63/484,878, filed on Feb. 14, 2023.
(60) Provisional application No. 63/580,243, filed on Sep. 1, 2023.
(60) Provisional application No. 63/618,232, filed on Jan. 5, 2024.

(51) Int. Cl. A61K 9/00; A61K 31/00; A61K 31/047; A61K 31/198; A61K 31/23; A61K 31/723; A61K 33/06; A61K 33/22; A61K 33/30; A61K 36/185; A61K 36/22; A61K 36/28; A61K 36/738; A61K 36/889; A61K 36/899; A61K 47/10; A61K 47/12; A61K 47/14; A61K 47/18; A61P 17/02; A61P 17/04; A61P 31/00

(52) U.S. Cl. CPC A61K 9/0014; A61K 31/047; A61K 31/198; A61K 31/23; A61K 31/658; A61K 31/723; A61K 33/06; A61K 33/22; A61K 33/30; A61K 36/22; A61K 36/28; A61K 36/5777; A61K 36/738; A61K 36/889; A61K 36/899; A61K 47/10; A61K 47/12; A61K 47/14; A61K 47/18; A61P 17/02; A61P 17/04; A61P 31/00

(57) ABSTRACT

The present disclosure provide, in part, formulations for topical use and methods of use thereof, for example, in treating skin disorders or conditions.

### CROSS REFERENCE

This application claims priority to U.S. Provisional Application No. 63/484,878 filed Feb. 14, 2023, U.S. Provisional Application No. 63/580,243 filed Sep. 1, 2023, and U.S. Provisional Application No. 63/618,232 filed Jan. 5, 2024, the contents of each of which are incorporated herein by reference.

### BACKGROUND

Skin diseases and conditions include, for example, atopic dermatitis (AD), commonly known as eczema, which is a multi-factorial autoimmune skin disease typically characterized by skin inflammation, pruritus (itchiness), redness, irritation and skin discomfort in general. The current standard-of-care includes corticosteroid-based topicals as the first line of treatment. For moderate to severe AD manifestations, biologics comprising monoclonal antibodies are also prescribed; for example, dupilumab and tralokinumab, which are IL blockers, to produce an anti-inflammatory effect. However, due to the risk of breakdown in the gastrointestinal (GI) tract, these drugs are administered by subcutaneous injection. Being immunosuppressants given systemically, they can give rise to a range of side effects ranging from infection at injection site to conjunctivitis and cold sores on lips and mouth. Other approved forms of treatment include JAK inhibitors (specifically JAK-1 and JAK-2 inhibitors, e.g., ruxolitinib (topical), abrocitinib (oral), upadacitinib (oral)), calcineurin inhibitors (e.g., tacrolimus (topical) and pimecrolimus (topical)), PDE-4 inhibitors (e.g., crisaborole (topical)). Each of these forms of treatments are not without its list of adverse reactions ranging from burning/stinging sensation at region of application to more serious side effects, such as, nausea, indigestion, diarrhea and headaches (for the JAK inhibitor class of drugs). Also, these are prescription drugs which necessitate visit to the physician's office and hinge upon payer reimbursement since out-of-pocket expenses can be significantly high. ranging around thousands of dollars every year per patient. Finally, depending on the class of drugs, many of the above are limited for pediatric usage, where some are approved for above 12 years old only and a few are available for over 2 years of age.

### SUMMARY

The present disclosure provide, in part, emulsified body butter formulations comprising multiple active ingredients, namely, botanicals and minerals, for topical use, targeting the pathways directly or indirectly, e.g., as indicated in atopic dermatitis, but exhibit high safety (no adverse side effects) and efficacy. Using a unique combination of polyphenols, amino acids, carboxylic acids, peptides and mineral salts, the formulations disclosed herein demonstrate a synergistic effect to break the irritation-itching-inflammation cycle, halt skin inflammation and initiate a long-lasting skin healing process, in a short time. In some embodiments, these effects are achieved by ensuring rapid absorption of the ointment and permeation into the affected skin region. The formulations described herein are oil-in-water emulsions where the aqueous phase carries the hydrophilic ingredients while the oil-phase comprises the mixture of different plant butters and plant oils. Because the external phase comprises water, and the internal phase is oil, the ointment is rapidly absorbed in under 5 minutes without leaving a greasy or oily trace on skin. Recently it was shown that a combination of polyphenolic compounds and amino acids trigger an immunomodulatory pathway leading to an anti-inflammatory effect in vivo (Liu J. et al, J. Agric. Food Chem. 2023, 71, 5, 2344-2355). In some embodiments, the body butter formulations described herein function on the basis of the aforementioned underlying principle of its action via a combined effect of the various polyphenols and amino acids present in its composition. In addition to anti-inflammatory effects, the formulations described herein are characterized by a unique mode of action associated with each of the individual ingredients, all of which act synergistically, for an enhanced clinical effect.

### BRIEF DESCRIPTION OF THE DRAWINGS

FIG. 1A shows a progression of treatment using ND01. FIG. 1B shows clinical evidence of wound healing of a moderate-sized open wound on application of NH02 twice daily.

FIG. 2A shows a certificate of analysis of NH02. FIG. 2B shows a preservative efficacy test performed on NH02.

FIG. 3A shows clinical data demonstrating significant mean reduction of 28% in IGA (P<0.05), using topical cream according to NH02. FIG. 3B shows clinical data demonstrating mean reduction of 19.3% in EASI score using topical cream of formulation NH02 of Example 2 (Test).

### DETAILED DESCRIPTION

Described herein, in part, are formulations for the treatment of skin diseases and disorders. Formulations described herein may comprise both hydrophilic and hydrophobic components.

Formulations described herein may be an emulsion comprising two immiscible liquids, which are finely dispersed in each other. For example, an oil-in water emulsion, where an oily phase (dispersed phase) is mixed into an aqueous phase (continuous phase). Formulations described herein may comprise one or multiple polyphenolic components such as: plant-based butters (e.g., mango, cacao) and oils (e.g., CBD, jojoba, MCT, amla, sunflower, sesame, coconut, sandalwood). Formulations described herein may have a hydrophilic phase, and may comprise colloidal oatmeal, echinacea extract, and rose water.

In an aspect, provided herein are formulations comprising:
butter selected from the group consisting of shea butter, mango butter, cacao butter, olive butter, hemp seed butter, almond butter, cashew butter, pistachio butter, macadamia butter, and babassu butter, or a combination thereof;
cannabidiol (CBD) oil or hemp seed oil;
jojoba oil; and
medium chain triglyceride (MCT) oil.

In some embodiments, provided herein are formulations comprising:
about 5% to about 35% of butter, wherein the butter is selected from the group consisting of shea butter, mango butter, cacao butter, olive butter, hemp seed butter, almond butter, cashew butter, pistachio butter, macadamia butter, and babassu butter, or a combination thereof;
about 1% to about 5% of cannabidiol (CBD) oil or hemp seed oil;
about 1% to about 10% of jojoba oil; and
about 5% to about 17% of medium chain triglyceride (MCT) oil.

In some embodiments, provided herein are formulations comprising:
about 5% to about 17% of butter, wherein the butter is selected from the group consisting of shea butter, mango butter, cacao butter, olive butter, hemp seed butter, almond butter, cashew butter, pistachio butter, macadamia butter, and babassu butter, or a combination thereof;
about 1% to about 5% of cannabidiol (CBD) oil or hemp seed oil;
about 1% to about 10% of jojoba oil; and
about 5% to about 17% of medium chain triglyceride (MCT) oil.

In some embodiments, provided herein are formulations comprising:
about 5% to about 17% of butter, wherein the butter is selected from the group consisting of shea butter, mango butter, cacao butter, olive butter, hemp seed butter, almond butter, cashew butter, pistachio butter, macadamia butter, and babassu butter, or a combination thereof;
about 1% to about 5% of cannabidiol (CBD) oil;
about 1% to about 10% of jojoba oil; and
about 5% to about 17% of medium chain triglyceride (MCT) oil.

In some embodiments, provided herein are formulations comprising:
about 5% to about 17% of mango butter;
about 5% to about 17% of cacao butter;
about 1% to about 5% of cannabidiol (CBD) oil;
about 1% to about 10% of jojoba oil; and
about 5% to about 17% of medium chain triglyceride (MCT) oil.

In an aspect, provided herein are formulations comprising:
butter selected from the group consisting of shea butter, mango butter, cacao butter, olive butter, hemp seed butter, almond butter, cashew butter, pistachio butter, macadamia butter, and babassu butter, or a combination thereof;
amla oil;
sesame oil;
sunflower seed oil; and
coconut oil.

In some embodiments, provided herein are formulations comprising:
about 5% to about 35% of butter, wherein the butter is selected from the group consisting of shea butter, mango butter, cacao butter, olive butter, hemp seed butter, almond butter, cashew butter, pistachio butter, macadamia butter, and babassu butter, or a combination thereof;
about 1% to about 5% of amla oil;
about 1% to about 10% of sesame oil;
about 3% to about 17% of sunflower seed oil; and
about 1% to about 8% of coconut oil.

In some embodiments, provided herein are formulations comprising:
about 5% to about 17% of butter, wherein the butter is selected from the group consisting of shea butter, mango butter, cacao butter, olive butter, hemp seed butter, almond butter, cashew butter, pistachio butter, macadamia butter, and babassu butter, or a combination thereof;
about 1% to about 5% of amla oil;
about 1% to about 10% of sesame oil;
about 3% to about 17% of sunflower seed oil; and
about 1% to about 8% of coconut oil.

In some embodiments, provided herein are formulations comprising:
about 5% to about 17% of mango butter;
about 5% to about 17% of cacao butter;
about 1% to about 5% of amla oil;
about 1% to about 10% of sesame oil;
about 3% to about 17% of sunflower seed oil; and
about 1% to about 8% of coconut oil.

In some embodiments, formulations described herein may further comprise mineral salts.

In some embodiments, formulations described herein may further comprise zinc mineral salts. In some embodiments, formulations described herein further comprise about 0.5% to about 5% of magnesium mineral salts. Formulations described herein may comprise a Zn compound with an anionic component selected from the following, singularly or in combination: oxide, acetate, aspartate, ascorbate, methionate, cysteinate, hydroxide, borate, carbonate, cerium oxide, chloride, citrate, glycinate, glycinate-salicylate, glutamate, gluconate, glucoheptonate, phosphate, dimethicone phthalate, and dimethicone succinate.

In some embodiments, formulations described herein may further comprise magnesium mineral salts. In some embodiments, formulations described herein further comprise about 0.5% to about 5% of zinc mineral salts. Formulations described herein may comprises a Mg compound with an anionic component selected from the following, singularly or in combination: oxide, citrate, chloride, nitrate, glycinate, alginate, acetate, ascorbate, bromide, fluoride, aspartate, alginate, carbonate, hydride, hydroxide, cocoate, coco-sulfate, coceth-sulfate, lactate, laureth, myreth sulfate, taurate, threonate, myristate, palmitate, and oleth-sulfate.

In some embodiments, formulations described herein may further comprise boric acid. In some embodiments, formulations described herein further comprise about 0.1% to about 1% of boric acid.

Formulations described herein may further comprise colloidal oatmeal. Colloidal oatmeal is characteristically water insoluble, and at best can form a colloidal solution only. But particles of oatmeal larger than 0.5 mm in diameter settle down at the bottom of the beaker holding a colloidal oatmeal solution. These particles manifest as blobs in the final cream and as such can have an unpleasant sight and feel. Besides, colloidal oatmeal comprises large protein molecules which are typically in the high kDa molecular weight range. While they can be applied topically to soothe the outer skin, in order for them to perform a therapeutic function in treating eczema, they need to be able to permeate into the stratum corneum. It is well known that molecules larger than 500 Da in size are highly unlikely to permeate the skin under normal conditions. In order to ensure absorption of the colloidal oatmeal into the skin to perform it's therapeutic function, this is oatmeal is first hydrolyzed by thermal treatment, and then homogenized to significantly lower particle size to micron range. This creates a uniform stable colloidal solution. Larger particles which may still persist are filtered through passing the colloidal solution through a cheesecloth with a relatively large pore size ~200-500 um. In some embodiments, formulations described herein further comprise about 0.1% to about 3% of colloidal oatmeal. In some embodiments, formulations described herein further comprise about 0.1% to about 1% of colloidal oatmeal.

In some embodiments, formulations described herein may further comprise an amino acid. In some embodiments, formulations descried herein further comprise about 0.5% to about 3% of L-histidine.

In some embodiments, formulations described herein may further comprise glycerin. In some embodiments, formulations descried herein further comprise about 0.5% to about 10% of glycerin.

In some embodiments, formulations described herein may further comprise xanthan gum. In some embodiments, formulations descried herein further comprise about 0.1% to about 2% of xanthan gum.

In some embodiments, formulations described herein may further comprise Echinacea extract. In some embodiments, formulations descried herein further comprise about 0.1% to about 3% of Echinacea extract.

In some embodiments, formulations described herein may further comprise sandalwood oil. In some embodiments, formulations descried herein further comprise about 0.1% to about 0.5% sandalwood oil.

In some embodiments, formulations described herein may further comprise rose water. In some embodiments, formulations descried herein further comprise about 0.5 to about 10% of rose water.

In some embodiments, formulations described herein may further comprise hempseed oil. In some embodiments, formulations descried herein further comprise about 0.1 to about 10% of hempseed oil.

In some embodiments, formulations described herein comprise DI water. In some embodiments, formulations described herein comprise about 30% to about 50% of DI water.

Formulations described herein may comprise butters, waxes, and other hydrophobic components. Depending on the ambient temperature (if >20° C.), it may be necessary to incorporate butters with higher melting points (e.g., shea butter), in order to prevent the formulation from liquefying and transforming into a runny texture. Additionally, waxes (e.g. beeswax, carnauba wax or paraffin wax) in about 0.1 to about 15% w/w %, about 0.5% to about 15% w/w, about 1% to about 15% w/w, about 0.1 to about 10% w/w %, about 0.5% to about 10% w/w, about 1% to about 10% w/w, about 0.1 to about 5% w/w %, about 0.5% to about 5% w/w, or about 1% to about 5% w/w, may be incorporated into the formulation to help in raising melting point of the final product, and thereby impart thermal stability. In some embodiments, formulations described herein are thermally stable (e.g., withstand a wide ambient temperature range from 0° C. to about 55° C., without freezing, melting, or layer separation).

In some embodiments, formulations described herein further comprise a surfactant, an emulsifier, a thickener, or a wax, or a combination thereof.

In some embodiments, formulations described herein further comprise glyceryl stearate. In some embodiments, formulations described herein further comprise about 0.5% to about 3% of glyceryl stearate.

In some embodiments, formulations described herein further comprise PEG 100 stearate. In some embodiments, formulations described herein further comprise about 0.5% to about 3% of PEG 100 stearate.

In some embodiments, formulations described herein further comprise stearic acid. In some embodiments, formulations described herein further comprise about 0.5% to about 5% of stearic acid.

In some embodiments, formulations described herein further comprise cetearyl alcohol. In some embodiments, formulations described herein further comprise about 1% to about 4% of cetearyl alcohol.

In some embodiments, formulations described herein further comprise emulsifying wax. In some embodiments, formulations described herein further comprise about 3% to about 6% of emulsifying wax.

In some embodiments, formulations described herein further comprise sorbitan monooleate. In some embodiments, formulations described herein further comprise about 3% to about 8% of sorbitan monooleate.

In some embodiments, formulations described herein further comprise Candelilla wax. In some embodiments, formulations described herein further comprise about 0.1% to about 3% of Candelilla wax.

In some embodiments, formulations described herein further comprise a combination of phenoxyethanol and caprylyl glycol. In some embodiments, formulations described herein further comprise about 0.2% to about 1% of a combination of phenoxyethanol and caprylyl glycol.

In some embodiments, formulations described herein further comprise a pH balancer. In some embodiments, formulations described herein further comprise about 1% to about 4% of a pH balancer. In some embodiments, the pH balancer is triethanolamine.

A key aspect of formulation design when combining an oil phase with water phase, is to determine the amount of surfactant needed to solubilize the oil phase into the water phase, to form an oil-in-water emulsion, such that the combining phases are miscible without phase separation. In order to scientifically design a stable formulation, the ‘hydrophilic-lipophilic balance’ or HLB should be determined from the individual oil phase and surfactant components used. This effective HLB is then utilized to select the appropriate emulsifier blend. Effective HLB in this formulation design was computed to be in the range 7-10. An appropriate blend of at least one hydrophilic and one lipophilic emulsifier is employed to match the effective HLB. In some embodiments, the effective hydrophilic-lipophilic balance (HLB) is greater than or equal to 7 and less than or equal to 10. In some embodiments, the formulations comprise at least one hydrophilic emulsifier with an HLB value greater than or equal to 9 and one lipophilic emulsifier with an HLB value less than or equal to 5. In some embodiments, the effective hydrophilic-lipophilic balance (HLB) is greater than 7 and less than 10. In some embodiments, the formulations comprise at least one hydrophilic emulsifier with an HLB value greater than 9 and one lipophilic emulsifier with an HLB value less than 5.

Formulations described herein may be applied once to several times a day. Formulations described herein may be applied at least twice daily. Formulations described herein may be applied at least three times daily. Formulations described herein may be applied at least four times daily. Also, formulations described herein may be applied for one, two, three, or for several weeks. Formulations described herein may be applied for at least one week. Formulations described herein may be applied for at least two weeks. Formulations described herein may be applied for at least three weeks. Formulations described herein may be applied for at least four weeks. The formulated cream when applied topically up to 3 times per day, works to lower local skin inflammation significantly and rapidly, reduce skin thickening or lichenification, reduce redness and/or hyperpigmentation associated with eczema and rashes, stop itchiness almost immediately, and heals and moisturizes the skin, retaining moisture inside the skin (not outside) without an oily/greasy feel on the exterior skin surface.

Formulations described herein may be paraben-free, sulfate-free, formaldehyde-free, non-comedogenic, non-steroidal. In some embodiments, formulations described herein do not comprise any animal-derived components (“vegan”). In some embodiments, formulations described herein are is essentially free of heavy metals, immunosuppressants, paraffin wax, mineral oil, alcohol, parabens, sulfates, or formaldehyde, or a combination thereof.

Formulations described herein may be for the treatment of skin related diseases or conditions. Formulations described herein may be for the treatment of autoimmune related diseases. Formulations described herein may be for the treatment of atopic dermatitis (AD), commonly known as eczema. Formulations described herein may be for the treatment of dermatitis. In some embodiments, dermatitis is atopic dermatitis, seborrheic dermatitis, contact dermatitis, stasis dermatitis, nummular dermatitis, perioral dermatitis, neurodermatitis dermatitis, or radiation dermatitis, or a combination thereof. Formulations described herein may be for the treatment of pruritus (itch). In some embodiments, the pruritus is associated with diabetes, fungal infection, dermatitis, neuropathy, pruritogen, or allergy, or a combination thereof. Formulations described herein may be for the treatment of cuts or wounds. In some embodiments, the cuts or wounds are mild cuts or wounds, mild-to-moderate cuts or wounds, surgical cuts or wounds, desquamation, or ulcerative cuts or wounds, or a combination thereof. Formulations described herein may be for the treatment of erythema, rash, redness, or skin irritation, or a combination thereof. Formulations described herein may be for the treatment of skin inflammation, lichenification, thickening, scaling, or roughening, or a combination thereof. Formulations described herein may be for the treatment of different forms of psoriasis. Formulations described herein may be for the treatment of mild-to-moderate burns. Formulations described herein may be for the treatment of insect bites on skin. Formulations described herein may be for the treatment of dry skin. Formulations described herein may be used as an antiseptic. In some embodiments, formulations described herein are antimicrobial. Formulations described herein may be for the treatment of neuropathic pain.

In some embodiments, formulations described herein are non-comedogenic.

In some embodiments, formulations described herein are suitable for all skin types.

In some embodiments, formulations described herein are pH balanced at a pH range of about 4 to about 6.

In some embodiments, formulations described herein, upon administration, reduce investigator global assessment (IGA), eczema area and severity index (EASI), or visual analog scale (VAS) scores.

In some embodiments, formulations described herein regenerate epidermal layers.

In some embodiments, formulations described herein promote skin repair.

In some embodiments, formulations described herein trap moisture inside the skin, prevent loss of moisture arising from, e.g., evaporation. The formulations described herein thus help skin stay moisturized for a long duration of time.

In some embodiments, formulations described herein permeate inside the skin layers in about 1 to about 20 minutes, or about 5 to about 10 minutes, or about 6 to about 10 minutes. The formulations described herein thus provide instant soothing relief.

In some embodiments, formulations described herein permeate into deeper skin layers.

In some embodiments, formulations described herein are contained within a packaging that is opaque (e.g., to prevent or reduce entry of light to prevent or reduce photodegradation and/or oxidation of photosensitive components within the formulation). In some embodiments, inner surface (wall) of the packaging is inert or stable against acidic conditions.

### EXAMPLES

In order that the disclosure described herein may be more fully understood, the following examples are set forth. The synthetic and biological examples described in this application are offered to illustrate the compounds, pharmaceutical compositions, and methods provided herein and are not to be construed in any way as limiting their scope.

### Example 1

The active ingredients (and concentration range in w/w %), used in an exemplary formulation are as follows:

### ND01:

DI water 30-50%
Mango butter 5-17%
Cacao butter 5-17%
Cannabidiol (CBD) oil or hemp seed oil 1-5%
Jojoba oil 1-10%
Medium chain triglyceride (MCT) oil 5-17%
Magnesium mineral salts 0.5-5%
Boric acid 0.1-1%
Zinc mineral salts 0.5-5%
Colloidal Oatmeal 0.1-3%
L-histidine 0.5-3%
Glycerin 0.5-10%
Xanthan Gum 0.1-2%
Echinacea extract 0.1-3%
Sandalwood oil 0.1-0.5%
Rose water 0.5-10%

### Example 2

In another embodiment, to create a non-comedogenic version, the above formulation was modified to the following:

### NH01:

DI water 30-50%
Mango butter 5-17%
Cacao butter 5-17%
Amla oil 1-5%
Sesame seed oil 1-10%
Sunflower seed oil 3-17%
Coconut oil 1-8%
Magnesium mineral salts 0.5-5%
Boric acid 0.1-1%
Zinc mineral salts 0.5-5%
Colloidal Oatmeal 0.1-3%
L-histidine 0.5-3%
Glycerin 0.5-10%
Xanthan Gum 0.1-2%
Echinacea extract 0.1-3%
Sandalwood oil 0.1-0.5%
Rose water 0.5-10%

### NH02:

In another embodiment, to create a thermally resilient version, that can endure higher temperatures without melting or destabilizing/breakdown of emulsion, along with pH balance and preservative effect to protect against antimicrobial growth, the following additional ingredients were included to NH01 in the formulation design:

| | |
|---|---|
| Glyceryl Stearate SE | 0.5-3.0% |
| PEG 100 Stearate | 0.5-3.0% |
| Stearic acid | 0.5-5.0% |
| Cetearyl alcohol | 1-4.0% |
| Emulsifying Wax NF | 3-6.0% |
| Sorbitan Monooleate | 3-8.0% |
| Candelilla Wax | 0.1-3% |
| Phenoxyethanol (and) Caprylyl Glycol | 0.2-1% |
| Triethanolamine | 1-4.0% |

1. Polyphenolic components are heavily present in both the lipophilic phase: plant-based butters (mango, cacao) and oils (CBD, jojoba, MCT, amla, sunflower, sesame, coconut, sandalwood); and hydrophilic phase: colloidal oatmeal, echinacea extract and rose water. This ensures ample permeation not only in the outermost stratum corneum and epidermal layers (lipophilic-aided) but also in deeper dermal layers (hydrophilic-aided), in turn effecting a deeper multi-layer skin absorption.
2. The Mg compound's anionic components are selected from the following, singularly or in combination: oxide, citrate, chloride, nitrate, glycinate, alginate, acetate, ascorbate, bromide, fluoride, aspartate, alginate, carbonate, hydride, hydroxide, cocoate, coco-sulfate, coceth-sulfate, lactate, laureth, myreth sulfate, taurate, threonate, myristate, palmitate or oleth-sulfate.
3. The Zn compound's anionic components are selected from the following, singularly or in combination: oxide, acetate, aspartate, ascorbate, methionate, cysteinate, hydroxide, borate, carbonate, cerium oxide, chloride, citrate, glycinate, glycinate-salicylate, glutamate, gluconate, glucoheptonate, phosphate, dimethicone phthalate or dimethicone succinate.
4. Colloidal oatmeal is characteristically water insoluble, and at best can form a colloidal solution only. But particles of oatmeal larger that 0.5 mm in diameter settle down at the bottom of the beaker holding a colloidal oatmeal solution. These particles manifest as blobs in the final cream and as such can have an unpleasant sight and feel. Besides, colloidal oatmeal comprises large protein molecules which are typically in the high kDa molecular weight range. While they can be applied topically to soothe the outer skin, in order for them to perform a therapeutic function in treating eczema, they need to be able to permeate into the stratum corneum. It is well known that molecules larger than 500 Da in size are highly unlikely to permeate the skin under normal conditions. In order to ensure absorption of the colloidal oatmeal into the skin to perform it's therapeutic function, this oatmeal is first hydrolyzed by thermal treatment, and then homogenized to significantly lower particle size to micron range. This creates a uniform stable colloidal solution. Larger particles which may still persist are filtered through passing the colloidal solution through a cheesecloth with a relatively large pore size ~200-500 μm.
5. Depending on the ambient temperature (if >20° C.), it may be necessary to incorporate butters with higher melting points (e.g., shea butter), in order to prevent the cream from liquefying and transforming into a runny texture. Additionally, waxes (e.g., beeswax, carnuba wax or paraffin wax) in 1-15% w/w % may be incorporated into the formulation to help in raising melting point of the final product, and thereby impart thermal stability.
6. The formulated cream when applied topically up to 3 times per day, works to lower local skin inflammation significantly and rapidly, reduce skin thickening or lichenification, reduce redness and/or hyperpigmentation associated with eczema and rashes, stop itchiness almost immediately, and heals and moisturizes the skin. retaining moisture inside the skin (not outside) without an oily/greasy feel on the exterior skin surface.
7. No adverse effects were observed in the duration of topical application of the emulsified body butter.
8. Paraben-free, sulfate-free, formaldehyde-free, non-comedogenic, non-steroidal.

### NH03:

In another embodiment, to create a formulation without fragrance generating ingredients, sandalwood oil and rosewater are replaced by hemp seed oil as follows:
DI water 30-50%
Mango butter 5-17%
Cacao butter 5-17%
Amla oil 1-5%
Sesame seed oil 1-10%
Sunflower seed oil 3-17%
Coconut oil 1-8%
Magnesium mineral salts 0.5-5%
Boric acid 0.1-1%
Zinc mineral salts 0.5-5%
Colloidal Oatmeal 0.1-3%
L-histidine 0.5-3%
Glycerin 0.5-10%
Xanthan Gum 0.1-2%
Echinacea extract 0.1-3%
Hempseed oil 0.1-10%

### NH04:

In another embodiment, Aloe Vera was incorporated, albeit, unsuccessfully in the formulation leading to phase separation as follows:
DI Water 40%
Aloe vera extract 10%
CBD oil 1%
Echinacea extract 1%
Jojoba oil 5%
MCT oil 11%
Mango butter 9%
Cacao butter 6%
Sandalwood oil 2%
Magnesium salt 1%
Boric acid 1%
Zinc oxide 3%
Colloidal oatmeal 1%
Glycerin 5%
Xanthan gum 0.5%
L-histidine 0.5%
PEG-60 1%
Germal plus 0.5%
Emulsifying wax NF 2%

### Example 3: Successful POC with Clinical Evidence

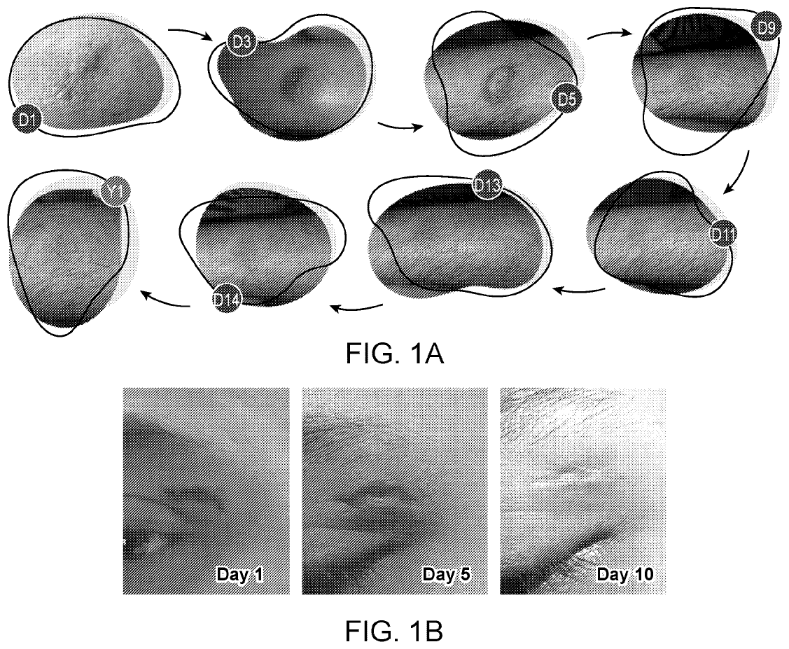

FIG. 1A shows a progression of treatment-moderate and chronic eczema lesion on left leg, before application of ND01 (D1) to rapid resolution of eczematic lesion after 2 weeks of daily twice application of ND01 from Day 1 (D1) to Day 14 (D14). Note skin lichenification, hyperpigmentation and pruritus have fully resolved. Hair follicular growth has returned to normal. Final progression to full and complete resolution is observed in the Year 1 (Y1) image without any flare-ups in the interim period of one year. FIG. 1B shows clinical evidence of wound healing of a moderate-sized open wound on application of NH02 twice daily as a standalone treatment from Day 1 to Day 10. Rapid closure of wound without the aid of stitch, regeneration of epidermis, antimicrobial and antiseptic effects, together with speedy reduction of inflammation can be observed.

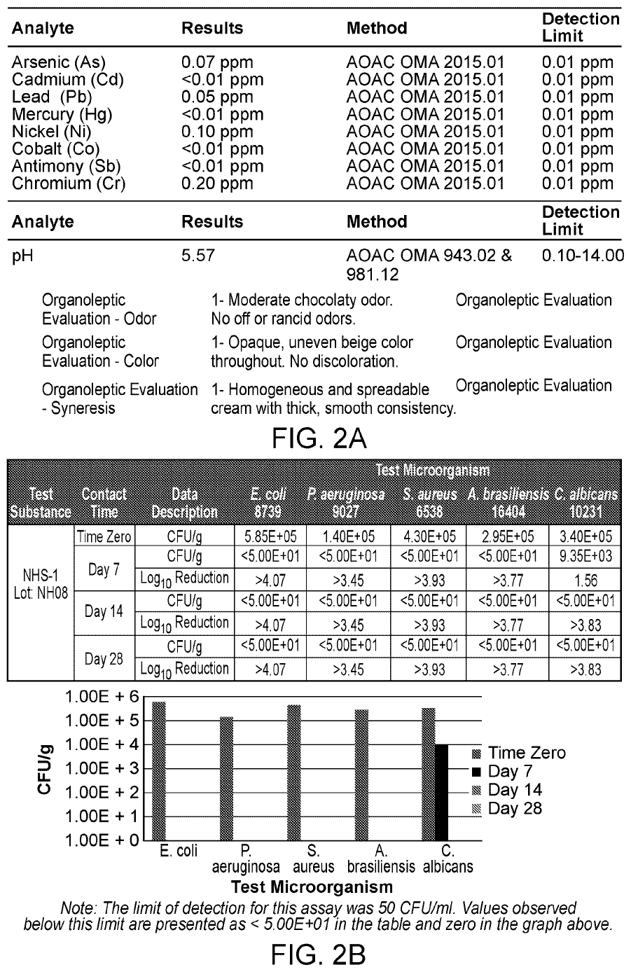

FIG. 2A shows a certificate of analysis of NH02. FIG. 2B shows a preservative efficacy test performed on NH02. Notably, the formulations exhibited antimicrobial effect demonstrated by the rapid log reduction of microbial titers from time zero to Day 7 and Day 14.

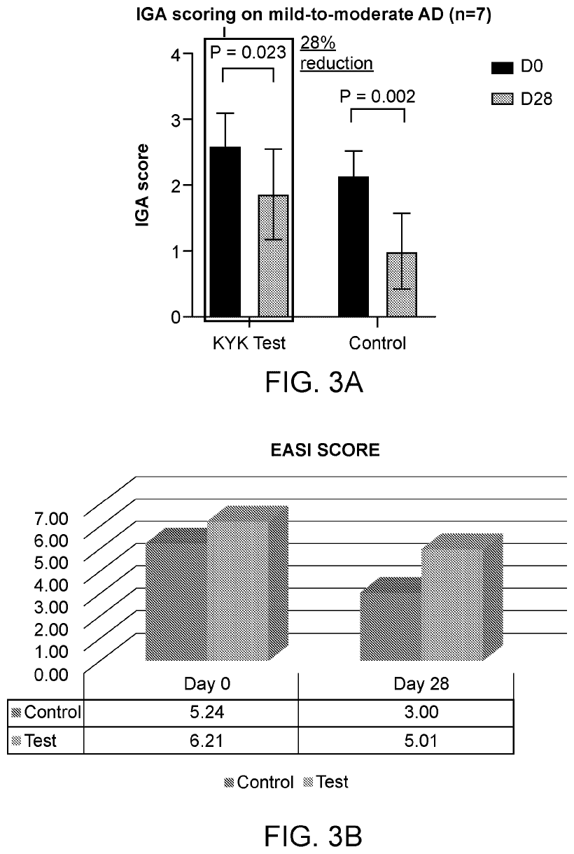

FIG. 3A shows clinical data demonstrating significant mean reduction of 28% in IGA (P<0.05), using topical cream according to NH02 of Example 2 for 28 days on patients with mild-to-moderate AD. Positive control: 0.1% Mometasone furoate topical. FIG. 3B shows clinical data demonstrating mean reduction of 19.3% in EASI score using topical cream of formulation NH02 of Example 2 (Test) for 28 days on patients with mild-to-moderate AD. Positive control: 0.1% Mometasone furoate topical.

What is claimed:

1. A formulation comprising:
about 5% to about 17% of butter;
wherein the butter is selected from the group consisting of shea butter, mango butter, cacao butter, olive butter, hemp seed butter, almond butter, cashew butter, pistachio butter, macadamia butter, and babassu butter, or a combination thereof;
about 1% to about 5% of cannabidiol (CBD) oil;
about 1% to about 10% of jojoba oil; and
about 5% to about 17% of medium chain triglyceride (MCT) oil.

2. The formulation of claim 1, further comprising about 0.5% to about 5% of magnesium mineral salts.

3. The formulation of claim 1 or 2, further comprising about 0.1% to about 1% of boric acid.

4. The formulation of any one of claims 1-3, further comprising about 0.5% to about 5% zinc mineral salts.

5. The formulation of any one of claims 1-4, further comprising about 0.1% to about 3% of colloidal oatmeal.

6. The formulation of any one of claims 1-5, further comprising about 0.5% to about 3% of L-histidine.

7. The formulation of any one of claims 1-6, further comprising about 0.5% to about 10% of glycerin.

8. The formulation of any one of claims 1-7, further comprising about 0.1% to about 2% of xanthan gum.

9. The formulation of any one of claims 1-8, further comprising about 0.1% to about 3% Echinacea extract.

10. The formulation of any one of claims 1-9, further comprising about 0.1% to about 0.5% sandalwood oil.

11. The formulation of any one of claims 1-10, further comprising about 0.5 to about 10% of rose water.

12. The formulation of any one of claims 1-11, further comprising about 30% to about 50% of DI water.

13. A formulation comprising:
about 5% to about 17% of butter,
wherein the butter is selected from the group consisting of shea butter, mango butter, cacao butter, olive butter, hemp seed butter, almond butter, cashew butter, pistachio butter, macadamia butter, and babassu butter, or a combination thereof;
about 1% to about 5% of amla oil;
about 1% to about 10% of sesame oil;
about 3% to about 17% of sunflower seed oil; and
about 1% to about 8% of coconut oil.

14. The formulation of claim 13, further comprising about 0.5% to about 5% of magnesium mineral salts.

15. The formulation of claim 13 or 14, further comprising about 0.1% to about 1% of boric acid.

16. The formulation of any one of claims 13-15, further comprising about 0.5% to about 5% zinc mineral salts.

17. The formulation of any one of claims 13-16, further comprising about 0.1% to about 3% of colloidal oatmeal.

18. The formulation of any one of claims 13-17, further comprising about 0.5% to about 3% of L-histidine.

19. The formulation of any one of claims 13-18, further comprising about 0.5% to about 10% of glycerin.

20. The formulation of any one of claims 13-19, further comprising about 0.1% to about 2% of xanthan gum.

21. The formulation of any one of claims 13-20, further comprising about 0.1% to about 3% Echinacea extract.

22. The formulation of any one of claims 13-21, further comprising about 0.1% to about 0.5% sandalwood oil.

23. The formulation of any one of claims 13-22, further comprising about 0.5 to about 10% of rose water.

24. The formulation of any one of claims 13-23, further comprising about 30% to about 50% of DI water.

25. The formulation of any one of claims of 1-24, wherein the effective hydrophilic-lipophilic balance (HLB) is greater than 7 and less than 10.

26. The formulation of any one of claims of 1-25, comprising at least one hydrophilic emulsifier with an HLB value greater than 9 and one lipophilic emulsifier of HLB value less than 5.

27. The formulation of any one of claims 1-26, further comprising a surfactant, an emulsifier, a thickener, or a wax, or a combination thereof.

28. The formulation of claim 27, wherein the formulation is thermally stable (e.g., withstands a wide ambient temperature range from 0° C. to about 55° C., without freezing, melting, or layer separation)

29. The formulation of any one of claims 1-28, further comprising about 0.5% to about 3% of glyceryl stearate.

30. The formulation of any one of claims 1-29, further comprising about 0.5% to about 3% of PEG 100 stearate.

31. The formulation of any one of claims 1-30, further comprising about 0.5% to about 5% of stearic acid.

32. The formulation of any one of claims 1-31, further comprising about 1% to about 4% of cetearyl alcohol.

33. The formulation of any one of claims 1-32, further comprising about 3% to about 6% of emulsifying wax.

34. The formulation of any one of claims 1-33, further comprising about 3% to about 8% of sorbitan monooleate.

35. The formulation of any one of claims 1-34, further comprising about 0.1% to about 3% of Candelilla wax.

36. The formulation of any one of claims 1-35, further comprising about 0.2% to about 1% of a combination of phenoxyethanol and caprylyl glycol.

37. The formulation of claim 36, wherein the combination is formaldehyde-free or paraben-free.

38. The formulation of any one of claim 1-37, further comprising about 1% to about 4% of a pH balancer.

39. The formulation of claim 38, wherein the pH balancer is triethanolamine.

40. The formulation of any one of claims 1-39, wherein the formulation is applied topically.

41. The formulation of any one of claims 1-40, wherein the formulation is for use in a skin related disease or condition.

42. The formulation of any one of claims 1-41, wherein the formulation is for use in treating dermatitis.

43. The formulation of claim 42, wherein the dermatitis is atopic dermatitis, seborrheic dermatitis, contact dermatitis, stasis dermatitis, nummular dermatitis, perioral dermatitis, neurodermatitis dermatitis, or radiation dermatitis, or a combination thereof.

44. The formulation of any one of claims 1-41, wherein the formulation is for use in treating pruritus (itch).

45. The formulation of claim 44, wherein the pruritus is associated with diabetes, fungal infection, dermatitis, neuropathy, pruritogen, or allergy, or a combination thereof.

46. The formulation of any one of claims 1-41, wherein the formulation is for use in treating cuts or wounds.

47. The formulation of claim 46, wherein the cuts or wounds are mild cuts or wounds, mild-to-moderate cuts or wounds, surgical cuts or wounds, desquamation, or ulcerative cuts or wounds, or a combination thereof.

48. The formulation of any one of claims 1-41, wherein the formulation is for use in treating erythema, rash, redness, or skin irritation, or a combination thereof.

49. The formulation of any one of claims 1-41, wherein the formulation is for use in treating skin inflammation, lichenification, thickening, scaling, or roughening, or a combination thereof.

50. The formulation of any one of claims 1-41, wherein the formulation is for use in treating different forms of psoriasis.

51. The formulation of any one of claims 1-41, wherein the formulation is for use in treating mild-to-moderate burns.

52. The formulation of any one of claims 1-41, wherein the formulation is for use in treating insect bites on skin.

53. The formulation of any one of claims 1-41, wherein the formulation is for use in treating dry skin.

54. The formulation of any one of claims 1-41, wherein the formulation is for use as an antiseptic.

55. The formulation of anyone of claims 1-54, wherein the formulation is antimicrobial.

56. The formulation of any one of claims 1-55, wherein the formulation does not comprise any animal-derived components (“vegan”).

57. The formulation of any one of claims 1-56, wherein the formulation is essentially free of heavy metals, immunosuppressants, paraffin wax, mineral oil, alcohol, parabens, sulfates, or formaldehyde, or a combination thereof.

58. The formulation of any one of claims 1-57, wherein the formulation is non-comedogenic.

59. The formulation of any one of claims 1-58, wherein the formulation is for all skin types.

60. The formulation of any one of claims 1-59, wherein the formulation is PH balanced at a pH range of about 4 to about 6.

61. The formulation of any one of claims 1-60, wherein the formulation reduces investigator global assessment (IGA), eczema area and severity index (EASI), or visual analog scale (VAS) scores.

62. The formulation of any one of claims 1-61, wherein the formulation regenerates epidermal layers.

63. The formulation of any one of claims 1-62, wherein the formulation promotes skin repair.

64. The formulation of any one of claims 1-63, wherein the formulation is applied daily.

65. The formulation of any one of claims 1-63, wherein the formulation is applied twice daily.

66. The formulation of any one of claims 1-63, wherein the formulation is applied daily for at least two weeks.

67. The formulation of any one of claims 1-63, wherein the formulation is applied daily for at least four weeks.

68. The formulation of any one of claims 1-63, wherein the formulation is applied daily for at least six weeks.

69. The formulation of any one of claims 1-63, wherein the formulation is applied twice daily for at least two weeks.

70. The formulation of any one of claims 1-63, wherein the formulation is applied twice daily for at least four weeks.

71. The formulation of any one of claims 1-63, wherein the formulation is applied twice daily for at least six weeks.

72. The formulation of any one of claims 1-63, wherein the formulation is applied at least three times daily.

73. The formulation of any one of claims 1-63, wherein the formulation is applied at least three times daily for at least two weeks.

74. The formulation of any one of claims 1-63, wherein the formulation is applied at least three times daily for at least four weeks.

75. The formulation of any one of claims 1-63, wherein the formulation is applied at least three times daily for at least six weeks.

76. The formulation of any one of claims 1-75, wherein the formulation traps moisture inside the skin.

77. The formulation of any one of claims 1-76, wherein the formulation permeates inside the skin layers in about 5 to about 10 minutes.

78. The formulation of any one of claims 1-77, for use in treating neuropathic pain.
